(19) United States
(12) Patent Application Publication
(10) Pub. No.: US 2025/0035609 A1
(43) Pub. Date: Jan. 30, 2025

(54) PLASTIC PART AND METHOD OF PROVIDING RECYCLING INFORMATION

(71) Applicant: Tetsuya YOSHIDA, Kanagawa (JP)

(72) Inventor: Tetsuya YOSHIDA, Kanagawa (JP)

(73) Assignee: Ricoh Company, Ltd., Tokyo (JP)

(21) Appl. No.: 18/768,674

(22) Filed: Jul. 10, 2024

(30) Foreign Application Priority Data
Jul. 24, 2023 (JP) 2023-119687

(51) Int. Cl. G01N 33/44

(52) U.S. Cl. CPC G01N 33/442

(57) ABSTRACT

A plastic part includes a display to display recyclability of the plastic part after use, and the display displays recycling information including a result of a shipping inspection of the plastic part based on shipping inspection data.

### CROSS-REFERENCE TO RELATED APPLICATIONS

This patent application is based on and claims priority pursuant to 35 U.S.C. § 119 (a) to Japanese Patent Application No. 2023-119687, filed on Jul. 24, 2023, in the Japan Patent Office, the entire disclosure of which is hereby incorporated by reference herein.

### BACKGROUND

### Technical Field

Embodiments of the present disclosure relate to a plastic part and a method of providing recycling information.

### Related Art

In the related art, a plastic part including recycling information for determining the recyclability of the plastic part after use is known.

For example, in the related art, there is a product such as a television or an air conditioner indoor unit including a part made of a thermoplastic resin (i.e., plastic part) that displays the recycling information for determining the recyclability of the plastic part after use. The recycling information is information evaluated based on the strength of a remolded plastic part to be obtained when the plastic part is recycled. Specifically, the strength of a plastic part obtained by remolding the plastic part is evaluated by using a tensile test, a bending test, or an Izod impact strength test.

### SUMMARY

According to an embodiment of the present disclosure, a plastic part includes a display to display recyclability of the plastic part after use, and the display displays recycling information including a result of a shipping inspection of the plastic part based on shipping inspection data.

According to an embodiment of the present disclosure, a method of providing recycling information includes displaying recycling information on a plastic part after use, and the recycling information includes a result of a shipping inspection of the plastic part based on shipping inspection data.

### BRIEF DESCRIPTION OF THE DRAWINGS

A more complete appreciation of embodiments of the present disclosure and many of the attendant advantages and features thereof can be readily obtained and understood from the following detailed description with reference to the accompanying drawings, wherein:

FIG. 1 is a schematic diagram illustrating an overall configuration of an image forming apparatus using plastic parts according to an embodiment of the present disclosure;

FIG. 2 is a diagram illustrating the image forming apparatus of FIG. 1 and plastic parts as exterior parts separated from the image forming apparatus;

FIG. 3 is a flowchart illustrating a process from manufacturing to recycling of plastic parts;

FIG. 4 is a diagram illustrating a foreign substance appearing on the surface of a plastic part;

FIG. 5 is a diagram illustrating a plastic part and the size in error measurement; and

FIG. 6 is a perspective view of a plastic part including a display of recycling information.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described below. As used herein, the singular forms “a,” “an,” and “the” are intended to include the plural forms as well, unless the context clearly indicates otherwise.

According to embodiments of the present disclosure, the manufacturing cost of the plastic part can be reduced as compared with the case where the evaluation process of the plastic part is conducted to obtain the recycling information added to the plastic part in addition to the manufacturing and shipping processes.

Embodiments of the present disclosure will be described below with reference to the drawings. In the present embodiment, the plastic part is described as an example of a plastic part used in an image forming apparatus. However, the plastic part can be also used for a plastic part used in any products other than the image forming apparatus. In particular, the plastic part can be used for a product that requires design and strength and can display a mark. Specific examples of the product to which the plastic part according to the present embodiment can be applied include household electric appliances such as air conditioners, projectors, cameras, automobile components, building materials such as outdoor decks, furniture such as tables, lenses for illumination, or pipes for water pipes.

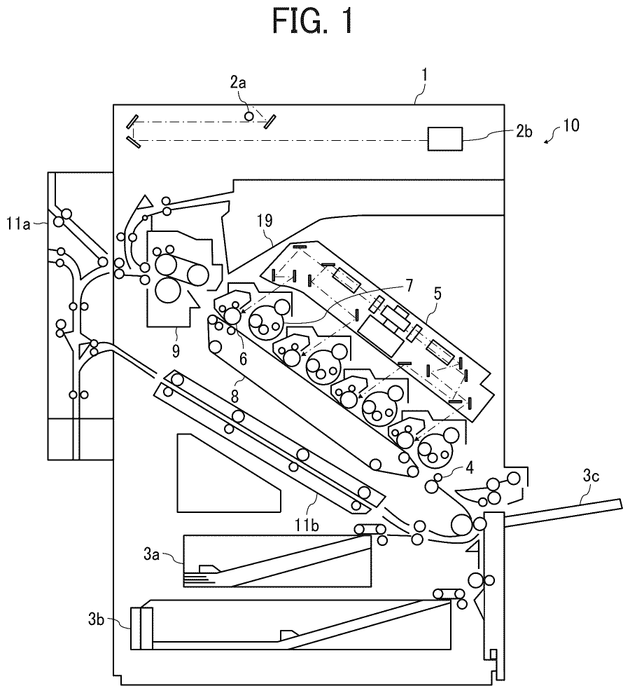

FIG. 1 is a schematic diagram illustrating an overall configuration of an image forming apparatus 10 using plastic parts according to an embodiment of the present disclosure. In the image forming apparatus 10 according to the present embodiment, a light source 2a emits light to irradiate a document placed on a document reading table 1 with the light, and a light receiver 2b reads reflection light from the document to acquire document information. The sheet (recording material or recording medium) fed from a sheet feeder 3a, 3b, or 3c is sent to a registration roller 4.

The optical writing unit 5 irradiates the photoconductor 6 with laser light according to the document information acquired by the light receiver 2b, and forms a latent image. The latent image formed on the photoconductor 6 is developed by a developing unit 7 as an image, and the image is transferred to the sheet sent from the registration roller 4 by a transfer unit 8. The sheet on which the image has been transferred is sent to an image fixing unit 9, and the image is fixed.

The sheet on which the image has been fixed is sent to an ejection sheet tray 19 or a reversing device 11a. The reversing device 11a reverses the direction of the sheet sent to the reversing device 11a, and sends the sheet of which the direction has been reversed to the reversing device 11b. The reversing device 11b sends the sheet to the registration roller 4 again, and an image is formed on the back surface of the sheet.

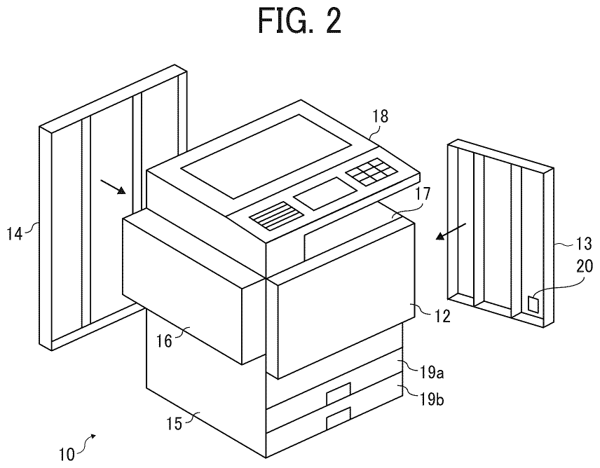

FIG. 2 is a diagram illustrating the image forming apparatus 10 and exterior parts, which are the plastic parts used in the image forming apparatus 10, separated from the image forming apparatus 10. The exterior of the image forming apparatus 10 according to the present embodiment includes exterior parts 12 to 18 and sheet accommodating parts (sheet feed trays 19a and 19b) that are made of a resin material (plastic material). In addition, these plastic parts are typically less likely to be damaged due to deformation, deterioration, or chipping, and are often large in size. Thus, these plastic parts are suitable for reuse and recycling.

The plastic parts such as the exterior parts 12 to 18 of the image forming apparatus 10 and the exteriors of the sheet accommodating parts (sheet feed trays 19a and 19b) include recycling information for determining the recyclability of the plastic parts after use. The recycling information may be electronically stored in a storage medium such as an integrated circuit (IC) chip included in a plastic part, or a display that indicates the recycling information may be included in a plastic part. In the present embodiment, as illustrated in FIG. 2, a recycling display 20 indicating recycling information is attached to a plastic part.

In the related art, the recycling information included in a plastic part uses the strength level obtained from the test result of a tensile test that uses a special jig and is conducted on a test piece obtained by remolding the plastic part. Such recycling information in the related art requires an additional process (i.e., a process of conducting various tests) separated from the manufacturing and shipping processes of the plastic part, and the manufacturing cost of the plastic part is increased.

The recycling information added to the plastic parts (exterior parts 12 to 18) of the image forming apparatus 10 according to the present embodiment is information determined based on the shipping inspection data used for determining whether the plastic parts pass the shipping inspection. In other words, in the present embodiment, the shipping inspection data used in the shipping inspection typically conducted to evaluate variations in the plastic part during the manufacturing process and detect a manufacturing error is used to determine the recycling information to determine the recyclability of the plastic part after use.

Accordingly, the recycling information can be determined from the shipping inspection data used in the shipping inspection conducted in manufacturing and shipping of the plastic part. Thus, the recycling information can be obtained without conducting an additional process (a process that conducts various tests) separated from the manufacturing and shipping processes of the plastic part, and the manufacturing cost can be reduced as compared with the cost in the related art.

Further, since the shipping inspection data is obtained for each shipped plastic part, the recycling information with high accuracy can be obtained for each plastic part as compared with the data obtained from another plastic part of the same type, for example, referred to as die inspection data.

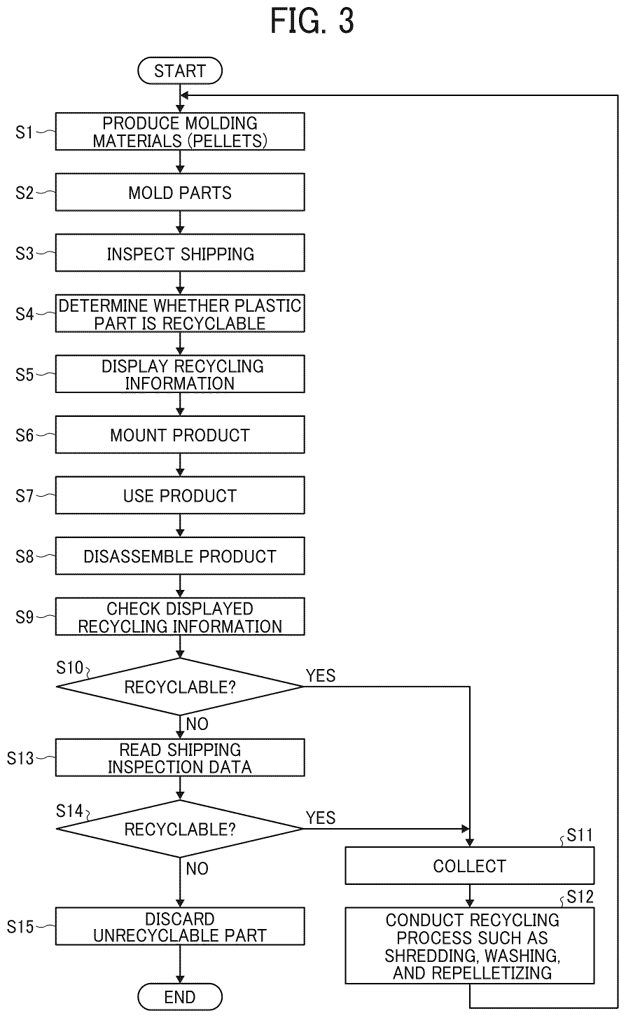

FIG. 3 is a flowchart illustrating a process from manufacturing to recycling of plastic parts (exterior parts 12 to 18). When the plastic parts of the image forming apparatus 10 are manufactured, a molding material (pellet) for plastic injection molding is produced (step S1). In this production process, for example, a new molding material and a recycled material collected are mixed to produce a molding material (pellet) as a produced molding material. In the part molding process, the plastic parts (exterior parts 12 to 18) are molded by an injection molding machine using the produced molding material (step S2).

The plastic parts (exterior parts 12 to 18) molded in this manner are sent to the shipping inspection process (step S3). In the shipping inspection process, it is inspected whether the mass production quality of the plastic parts can be acquired. The shipping inspection is preferably a non-destructive inspection (inspection that does not damage the parts and does not impair the shipping quality) of the plastic parts to be inspected. In the case of the non-destructive inspection, since the plastic parts to be shipped themselves can be inspected, the error of the inspection result is small, and the inspection cost can be decreased because there is no need to additionally prepare the evaluation parts or the test pieces.

Specifically, the shipping inspection includes, for example, appearance inspection that inspects appearance abnormality due to contamination of impurities in a molding material and temperature fluctuation during part molding. In the appearance inspection, for example, a color difference and the amount of foreign substance that appears in the appearance of the plastic part are inspected. Also, for example, there is a size measurement of mass production management that measures size abnormality caused by shrinkage fluctuation after molding the plastic part due to the influence of molding condition adjustment, and wear of a mold of the molding machine. In the size measurement of mass production management, for example, it is checked whether the plastic part to be inspected is molded in a predetermined size.

According to the present embodiment, a process that adds the recycling information to the plastic part is conducted. However, the recycling information according to the present embodiment is determined based on the shipping inspection data used in the shipping inspection process of the plastic part, as described above. Thus, in the present embodiment, a data acquisition process for determining the recycling information is not required separately from the shipping inspection process.

The shipping inspection data used for determining the recycling information according to the present embodiment includes data obtained by the appearance inspection described above, or data obtained by the size measurement of mass production management, and whether or not the resource recycling is possible is determined based on these shipping inspection data (step S4).

For example, in terms of the color difference of the appearance of the plastic part, which is data obtained in the appearance inspection, a plastic part (recycled plastic part) including a recycled material may be burned due to an increase in thermal history or may have a difference in color due to mixing of recycled materials of different colors. In particular, a difference in color remarkably appears in a transparent plastic part and a white plastic part. Accordingly, a plastic part having a large difference in color from the original color (color of a plastic part manufactured only from a new material) is determined to be deteriorated and thus to be unrecyclable.

As a method of determining whether the plastic part is recyclable based on the color difference data (shipping inspection data), for example, the color of a master sample made of only a new material is used as a reference, and the range of the color difference at which the pass level (the plastic part is recyclable) is set as a determination condition. If the plastic part to be inspected is within the range of the color difference of the determination condition, it is determined that the plastic part is allowed to recycle (recyclable), and if the plastic part is out of the range of the color difference of the determination condition, it is determined that the plastic part is not allowed to recycle (unrecyclable).

As the color difference data, for example, color coordinates in the L*a*b* color system can be used. Specifically, the distance ΔE between the color coordinates of the master sample and the color coordinates of the plastic part to be inspected can be obtained from Equation 1 below from the differences ΔL, Δa, and Δb in each direction of the color coordinates of the master sample and the color coordinates of the plastic part. This distance ΔE can be used as the color difference data used for determining whether the plastic part is recyclable.

$$Δ E = {[ {( Δ {L}^{*} )}^{2} + {( Δ {a}^{*} )}^{2} + ( Δ {b}^{*} ]}^{1 / 2} \qquad Equation 1$$

The color difference data may be measured by, for example, a handy color difference measurement device so as to be in contact with the plastic part to be inspected.

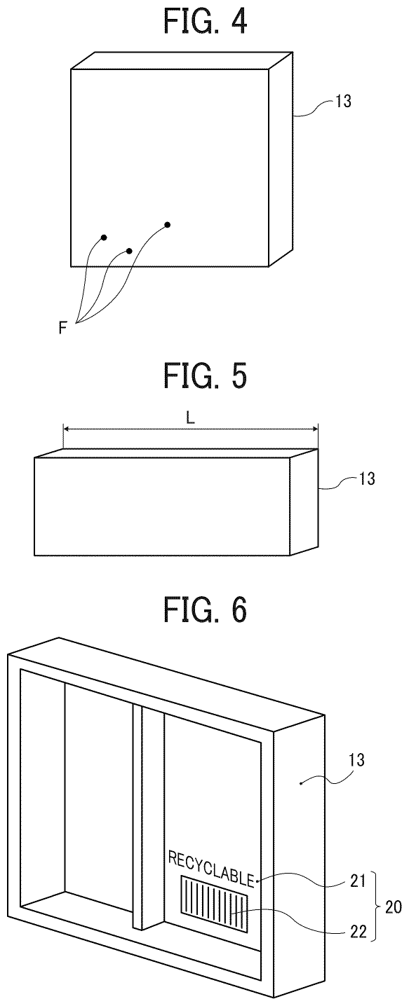

In addition, in terms of the amount of foreign substance in the appearance of the plastic part, which is data obtained in the appearance inspection, in a plastic part (recycled plastic part) including a recycled material, impurities mixed at the time of collection may appear as a foreign substance F on the surface of the plastic part as illustrated in FIG. 4. The larger the size of the foreign substance F appearing on the surface of the part is or the larger the amount of the foreign substance F is, the more the number of recycling times is and the more the amount of the impurity mixed is. Thus, a part in which the size or the amount of the foreign substance F appearing on the surface of the plastic part exceeds the determination reference is determined to be unrecyclable.

As a method for determining recyclability of the plastic part by the measurement data (shipping inspection data) of the size or the amount of the foreign substance F, for example, the size of the foreign substance F appearing on the surface of the plastic part is measured, or the number of the foreign substance F (density) appearing on the surface of the plastic part is counted. Based on the predetermined reference (determination reference), the recyclability of the plastic part is determined. For example, the image of the surface of the plastic part is captured by a camera, and the captured data of the image is processed to measure the size of the foreign substance and count the number of the foreign substance (density). In a simple manner, the size of the foreign substance may be determined by comparing the foreign substance with a scale for size determination (e.g., a chart for measurement of foreign substances), or the number of foreign substance in the area may be counted by applying a scale of a specified area to the part.

In Table 1 below, as an example, the determination condition (determination reference) and the determination results of the size and the amount of the foreign substance F are listed.

TABLE 1

| | NUMBER (DENSITY) OF | DETERMINATION |
|---|---|---|
| SIZE | FOREIGN SUBSTANCE | RESULT |
| >1 mm² | ONE OR MORE WITHIN | UNRECYCLABLE |
| | THE SAME PART | |
| | ZERO WITHIN THE SAME | RECYCLABLE |
| | PART | |
| >0.2 mm² & | TWO OR MORE WITHIN | UNRECYCLABLE |
| ≤1 mm² | 30 MM IN RADIUS | |
| | ONE OR LESS WITHIN | RECYCLABLE |
| | 30 MM IN RADIUS | |
| ≤0.2 mm² | NUMBER (DENSITY) | RECYCLABLE |
| | IGNORABLE | |

In addition, in terms of the size of the plastic parts, which are data obtained in the size measurement of mass production management, the plastic part (recycled plastic part) including the recycled material may contain impurities at the time of collection, or may contain materials that are the same as the new materials but different in grade from the new materials. Thus, the shrinkage rate at the time of molding differs between a plastic part including a recycled material and a plastic part containing only a new material. Accordingly, when the molding is conducted by the same mold, a difference occurs in the size of the plastic part after molding. The greater the amount of impurity or other grade of material is, the greater the difference in the size of the molded part is. Thus, a plastic part that has a difference in size exceeding the determination reference is determined to be unrecyclable.

As a method for determining whether a plastic part is recyclable based on the measurement data of the part size (shipping inspection data), for example, it is effective to use measurement data of the overall part length L (length of the side having the longest length of the part) as illustrated in FIG. 5 because the difference in shrinkage becomes more significant as the part size increases. For this measurement, a measurement method often used as a method for evaluating the quality check (molding variation) of a part in mass production can be applied, and the measurement can be simply conducted by a measurement jig such as a caliper.

It is determined whether the plastic part is recyclable in accordance with a predetermined determination condition (determination reference) based on the measured data of the part size. As the determination condition, for example, a tolerance as a determination reference is set for the overall part length to be measured. If the plastic part to be inspected is within the tolerance of the determination condition, it is determined that the plastic part is allowed to recycle (recyclable), and if the plastic part is out of the tolerance of the determination condition, it is determined that the plastic part is not allowed to recycle (unrecyclable).

As described above, it is determined whether the plastic part is recyclable based on the shipping inspection data, and the process that adds the display 20 of recycling information is conducted (step S5). In this process, for example, the display 20 that indicates the result of the process of determining whether the plastic part is recyclable (step S4) is added to each plastic part by marking.

Any displaying method may be used for the display 20 as long as it can be added after the plastic part is molded. For example, as the displaying method, laser marking, silk screen printing, pad printing, or decal application may be used. In addition, in the case where the display 20 cannot be added to a portion of the plastic part that is visible from the outside at the time when the plastic part is mounted in consideration of aesthetic appearance, the display 20 may be added to the plastic part with an invisible ink that is difficult to recognize in the visible light region but becomes recognizable when irradiated with black light (high-frequency ultraviolet light).

As described above, the plastic parts (exterior parts 12 to 18) to which the display 20 of the recycle information is added as described above are sent to the assembly process of the image forming apparatus 10 as a product on which the plastic parts are mounted (step S6), the image forming apparatus 10 is shipped, and the image forming apparatus 10 shipped is used by a user (step S7).

The image forming apparatus 10 after use is collected and sent to the disassembly process (step S8). In the disassembly process, the operator disassembles the image forming apparatus 10 after use and extracts plastic parts (exterior parts 12 to 18) for the image forming apparatus 10. The operator checks the display 20 of the recycling information (step S9) added to the plastic part extracted from the image forming apparatus 10, and determines whether the plastic part is recyclable based on the recycling information (step S10).

FIG. 6 is a perspective view of the plastic part (exterior part 13) including the display 20 of recycling information. In the present embodiment, in the checking process of the display of the recycling information (step S9), the operator (human) determines whether the plastic part is recyclable. In this case, the display 20 of the recycling information is preferably a display that allows the operator to immediately determine whether the plastic part is recyclable.

For example, in the determining process of the recycling (step S4), when the plastic part is determined to be recyclable, a letter display 21 “RECYCLABLE” is added to the plastic part as illustrated in FIG. 6. By contrast, when the plastic part is determined not to be recyclable (i.e., unrecyclable), a letter display 21 of “UNRECYCLABLE” is added to the plastic part. The display that can be immediately recognized by a human is not limited to the letter display 21, and, for example, symbol displays indicating recyclable and unrecyclable may be used. Further, a color symbol or mark may be used. For example, a blue mark indicates that the plastic part is recyclable, a red mark indicates that the plastic part is unrecyclable.

The recycling information added to the plastic part is not limited to recycling information that is a human-recognizable display. For example, as illustrated in FIG. 6, a code image 22 that is coded and can be read by a machine may be added to the plastic part. As the code image 22, a one-dimensional code image such as a barcode, or a two-dimensional code image can be used. The recycling information may be added to the plastic part so as to be electronically stored in a storage medium such as an integrated circuit (IC) chip. When the recycling information is added to the plastic part so as to be a machine-readable form, the determination of a recyclable plastic part can be suitably automated by machine reading.

The recycling information that displays the result of the determining process (step S4) is not limited to two choices of “RECYCLABLE” that indicates that the plastic part is recyclable and “UNRECYCLABLE” that indicates that the plastic parti is unrecyclable, but may be three or more choices. For example, in addition to “RECYCLABLE” and “UNRECYCLABLE”, a third choice of “DETERMINE AT THE TIME OF RECYCLING” may be added. In other words, the third choice is “indetermination” information. In this case, the recyclability of the plastic part to which the recycling information of “DETERMINE AT THE TIME OF RECYCLING” has been added is determined in accordance with a new reference at the time (step S10).

In Table 2 below, as an example, the determination conditions (determination reference) and the determination results of the size and the amount of the foreign substance F are listed.

TABLE 2

| | NUMBER | | |
|---|---|---|---|
| | (DENSITY) OF | | |
| | FOREIGN | LETTER | DETERMINATION |
| SIZE | SUBSTANCE | DISPLAY | RESULT |
| >1 mm² | ONE OR MORE | A | UNRECYCLABLE |
| | WITHIN THE SAME | | |
| | PART | B | UNRECYCLABLE |
| >0.2 mm² | THREE OR MORE | | |
| & ≤1 mm² | WITHIN 30 MM | | |
| | IN RADIUS | | |
| | TWO WITHIN | C | DETERMINE AT |
| | 30 MM IN RADIUS | | THE TIME OF |
| | | | RECYCLING |
| | ONE OR LESS | D | RECYCLABLE |
| | WITHIN 30 MM | | |
| | IN RADIUS | | |
| ≤0.2 mm² | NUMBER (DENSITY) | E | RECYCLABLE |
| | IGNORABLE | | |

In the example listed in Table 2, the plastic parts are classified into five categories depending on the determination conditions (determination reference) for the size and the amount of the foreign substance F, and five types of letter display A to E corresponding to the respective categories are added to the plastic parts as display of the recycling information. At this time, the letters A and B correspond to the determination result of “UNRECYCLABLE”, the letter C corresponds to the determination result of “DETERMINE AT THE TIME OF RECYCLING”, and the letters D and E correspond to the determination result of “RECYCLABLE”. Instead of the five types of letter display A to E, for example, symbolic displays of “circle, square, triangle, reverse triangle, and cross” or color marks of “blue, red, yellow, green, and brown” may be used.

The recycling information added to the plastic part may include not only the determination result in the determination of recycling (step S4), but also the shipping inspection data used in the determination or data (such as data indicating the quality level) generated from the shipping inspection data. If such data is added to the plastic part as recycling information, when the determination reference is changed after the shipping of the plastic part (at the time of the determination of the recycling (step S4)), the recycling (step S10) can be determined in accordance with a new determination reference. Depending on the product in which the plastic parts are mounted, many of the plastic parts will be sent to the recycling process after a period of five or more years from the shipping. It is likely to widen the range of qualities of recyclable plastic parts as the recycling technology advances during that period. Thus, it is beneficial in reducing the discarding rate of the plastic parts to add the shipping inspection data used in the determination of recycling (step S4) to the plastic part as the recycling information so that recyclability can be determined (step S10) in accordance with the latest determination reference.

In the present embodiment, the recycling information added to the plastic part includes the shipping inspection data used in the determination process of recycling (step S4). More specifically, as illustrated in FIG. 6, a code image 22 indicating the shipping inspection data (e.g., surface color difference data, the amount of the foreign substance on the surface, and size data of the plastic part) is added to the plastic part. Accordingly, since the shipping inspection data for determining the recyclability of the plastic part can be obtained by reading the code image 22 using the code reader, it is easy to determine the recyclability of the plastic part in accordance with a new determination reference at the time of determining the recyclability of the plastic part (step S10).

In particular, in the present embodiment, in addition to the recycling information of “RECYCLABLE” and “UNRECYCLABLE”, the recycling information of “DETERMINE AT THE TIME OF RECYCLING” is also prepared, and can be added to the plastic part as the letter display 21. Accordingly, the code image 22 added to the plastic part with the letter display 21 of “DETERMINE AT THE TIME OF RECYCLING” can be read using the code reader, and the recyclability of the plastic part can be determined in accordance with the new determination reference. Thus, the operation becomes easier than the case where the code images 22 of all the plastic parts are read to determine the recyclability of the plastic part.

The plastic part determined to be recyclable (Yes in step S10) in the determination of recyclability (step S10) is collected (step S11) and sent to the recycling process (step S12). In the recycling process, the collected plastic parts are pulverized, washed, re-pelletized, and processed for reuse as a molding material (pellet) for a plastic part, and reused as a molding material (pellet) for a plastic part.

By contrast, the plastic part determined to be unrecyclable (No in step S10) are discarded (step S13). Specifically, the code image 22 added to the plastic part is read by a code reader, and shipping inspection data for determining the recyclability of the plastic part is obtained. The plastic part is determined whether to be recyclable in accordance with the latest determination reference at the present time based on the shipping inspection data obtained (step S14). In the case where the determination reference at the present time has not been changed since the time of shipping the plastic part, this process can be omitted.

The plastic part determined to be recyclable (Yes in step S14) in the determination of recyclability (step S10) is collected (step S11), sent to the recycling process (step S12), and reused as a molding material (pellet) for a plastic part. By contrast, the plastic part determined to be unrecyclable (No in step S14) is discarded (step S15).

As described above, according to the present embodiment, in the determination of the recyclability of the plastic part, since the shipping inspection data conducted as quality check of the mass production at the time of the part manufacturing is used, the additional evaluation process for determining the recyclability is not required. In addition, in the present embodiment, since the shipping inspection data used for determining whether the plastic part is recyclable is the non-destructive inspection data obtained by the non-destructive inspection such as the color difference, the amount of foreign substance, and the size measurement of mass production management, a specific test piece or jig is not required, and thus, the number of measurement process is small, and the cost can be decreased.

As described above, aspects and advantageous effect of the present disclosure are, for example, as follows.

### First Aspect

A plastic part (e.g., the exterior parts 12 to 18) includes a display to display recyclability of the plastic part after use, and the display displays recycling information including a result of a shipping inspection of the plastic part based on shipping inspection data. In the first aspect, the recycling information added to the plastic part is information determined based on the shipping inspection data used for determining whether the plastic part passes the shipping inspection.

Accordingly, the recycling information can be determined from the shipping inspection data used in the shipping inspection conducted in manufacturing and shipping of the plastic part.

Thus, the recycling information can be obtained without conducting an additional process (a process that conducts various tests only to determine the recycling information) separated from the manufacturing and shipping processes of the plastic part, and the manufacturing cost can be reduced as compared with the cost in the related art.

### Second Aspect

In the plastic part according to the first aspect, the shipping inspection data includes non-destructive testing data of the plastic part.

Accordingly, since the plastic part to be shipped as it is can be inspected, the error of the inspection result is small, and the inspection cost can be decreased because additional evaluation parts or test pieces are not required.

### Third Aspect

In the plastic part according to the second aspect, the shipping inspection data includes at least one of data of color difference of a surface of the plastic part, data of amount of foreign substance on a surface of the plastic part, or data of size difference of the plastic part.

According to such the shipping inspection data, the recycling information of the plastic part can be determined with high accuracy.

### Fourth Aspect

In the plastic part according to any one of the first to third aspects, the recycling information includes the shipping inspection data.

Accordingly, when the determination reference is changed after the plastic part is shipped, the recyclability of the plastic part can be determined in accordance with a new determination reference, using the shipping inspection data included in the recycling information added to the plastic part.

Accordingly, the discard rate of the plastic part can be decreased because the recyclability of the plastic part can be easily determined by the new determination reference.

### Fifth Aspect

In the plastic part according to any one of the first to fourth aspects, the display displays the recycling information including the shipping inspection data.

Accordingly, the recycling information can be rapidly grasped from the display added to the plastic part, and the recyclable plastic part can easily be selected.

### Sixth Aspect

In the plastic part according to the fifth aspect, the display displays recyclability of the plastic part after use of the plastic part, or un-recyclability of the plastic part after use of the plastic part.

Accordingly, the recyclability of the plastic part can be rapidly determined, and the recyclable plastic part can rapidly be selected.

### Seventh Aspect

In the plastic part according to the fifth or sixth aspect, the display includes a code image coding the recycling information.

Accordingly, since the recycling information is easy to obtain by machine-reading, at least a portion of the determination process of a recyclable plastic part can be suitably automated.

### Eighth Aspect

A method of providing recycling information includes displaying recycling information on a plastic part after use, and the recycling information includes a result of a shipping inspection of the plastic part based on shipping inspection data.

Accordingly, the recyclability of the plastic part can be determined based on the recycling information determined the shipping inspection data used in the shipping inspection conducted in the manufacturing and shipping processes of the plastic part. Thus, the low-cost determination of the recyclability of the plastic part can be achieved as compared with the determination in the related art.

### Ninth Aspect

The method of providing recycling information according to the eighth aspect further includes displaying the recycling information including recyclability, un-recyclability, or indetermination of the plastic part after use, and selecting the recyclability or the un-recyclability based on the shipping inspection data when the indetermination is displayed in the displaying. The recycling information includes the shipping inspection data.

Accordingly, when the determination reference is changed after the plastic part is shipped, the recyclability of the plastic part can be determined in accordance with a new determination reference, using the shipping inspection data included in the recycling information added to the plastic part.

Accordingly, the discard rate of the plastic part can be decreased because the recyclability of the plastic part can be easily determined by the new determination reference. In particular, when the recycling information added to the plastic part does not include recyclability information and un-recyclability information (i.e., includes un-determination information), the recyclability or the un-recyclability is selected based on the shipping inspection data included in the recycling information.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention. Any one of the above-described operations may be performed in various other ways, for example, in an order different from the one described above.

1. A plastic part comprising: a display to display recyclability of the plastic part after use, and the display displaying recycling information including a result of a shipping inspection of the plastic part based on shipping inspection data.

2. The plastic part according to claim 1,
wherein the shipping inspection data includes non-destructive testing data of the plastic part.

3. The plastic part according to claim 2,
wherein the shipping inspection data includes at least one of: data of color difference of a surface of the plastic part; data of amount of foreign substance on a surface of the plastic part, or data of size difference of the plastic part.

4. The plastic part according to claim 1,
wherein the recycling information includes the shipping inspection data.

5. The plastic part according to claim 1,
wherein the display displays the recycling information including the shipping inspection data.

6. The plastic part according to claim 5,
wherein the display displays: recyclability of the plastic part after use of the plastic part, or un-recyclability of the plastic part after use of the plastic part.

7. The plastic part according to claim 5,
wherein the display includes a code image coding the recycling information.

8. A method of providing recycling information comprising displaying recycling information on a plastic part after use, the recycling information including a result of a shipping inspection of the plastic part based on shipping inspection data.

9. The method of providing recycling information according to claim 8, further comprising:
displaying the recycling information including recyclability, un-recyclability, or indetermination of the plastic part after use, the recycling information including the shipping inspection data; and
selecting the recyclability or the un-recyclability based on the shipping inspection data when the indetermination is displayed in the displaying.
